(19) United States
(12) Patent Application Publication
(10) Pub. No.: US 2002/0064881 A1
(43) Pub. Date: May 30, 2002

(54) Method for automatically storing and reprocessing patient specimen's in an automatic clinical analyzer

(76) Inventors: William Jackson Devlin, Lincoln University, PA (US); David R. Thompson, Kennett Square, PA (US); Cynthia G. Tymeson, Lincoln University, PA (US)

Correspondence Address: Dade Behring Inc., Legal Dept. -Patents, Attn: Lois K. Ruszala, 1717 Deerfield, Rd., #778, Deerfield, IL, 60015-0778, US

(21) Appl. No.: 09/827,045

(22) Filed: Apr. 5, 2001

Related U.S. Application Data
(63) Continuation-in-part of application No. 09/725,621, filed on Nov. 30, 2000.

(51) Int. Cl. G01N 35/00; G06F 19/00; G01N 33/48

(52) U.S. Cl. 436/43; 702/19

(57) ABSTRACT

A method to additionally test a patient's specimen using an analyzer some period of time after initial tests on an aliquot of the patient's specimen are completed by retaining the aliquot of the patient's specimen within the analyzer or by retaining another aliquot of the patient's specimen within the analyzer for a period of time.

### FIELD OF THE INVENTION

The present invention relates to an automated clinical analyzer for processing liquid samples, particularly for processing biological fluids such as urine, blood serum, plasma, cerebrospinal fluid and the like. In particular, the present invention provides a method and apparatus to automatically reprocess a sample aliquot retained in storage for a predetermined period of time on an automated clinical analyzer.

### BACKGROUND OF THE INVENTION

Fully automated diagnostic analyzers are commercially available to perform chemical, and immunoassaying of biological fluids such as urine, blood serum, plasma, cerebrospinal fluid and the like. Generally, reactions between an analyte to be measured in the sample and reagents used during the assay result in generating some sort of signal that can be measured by the instrument, and from this signal the concentration of analyte in the patient sample may be calculated. Diagnostic analyzers generally employ a large number of various processing stations, where operations such as sample and reagent addition, mix, wash and separate, are performed.

Heterogeneous immunoassays are popularly used because their versatility allows both large and small sized analytes to be measured and also because a physical separation step eliminates most interfering substances thereby providing for a higher sensitivity. Heterogeneous assays are either competitive immunoassays or sandwich immunoassays and in both types of such immunoassays, considerable resources and time are required to achieve a sufficiently high degree of washing so as to eliminate interfering constituents and prevent spurious assay results. The degree to which this is achieved in an automated analyzer is an important contributor to the competitive performance of the analyzer.

Another important contributor to maintaining a high throughput of automatic analyzers is the ability to process a plurality of samples through a variety of the different assay process steps that are needed before the signal measurement step may be undertaken in a time-effective manner. In the design of new automatic analyzers, in particular those involving complex “sandwich” heterogeneous immunoassays which often require about 30-40 separate processing operations, the ability to maintain a high throughput is an important performance criteria.

Finally, a significant amount of effort is undertaken to insure that the accuracy of results obtained using automated clinical analyzers is not adversely affected by the various reagents and sample analysis procedures employed in performing different assay process steps, measuring techniques in particular.

The extensive efforts made to achieve these objectives are made clear by an examination of various aspects of modern analyzers.

U.S. Pat. No. 5,981,296 relates to a method for stabilizing particle reagents suitable for use in turbidimetric immunoassays are disclosed. The stabilized particle reagents contain functionalized polymer particles in which the surface of the particle has been modified with a molecular surface modifier. The stabilized particle reagents are resistant to premature or spontaneous aggregation during preparation or storage.

U. S. Pat. No. 5,827,744 pertains to a method for cleaning a liquid sample probe in which the probe is positioned within a washing chamber inside a wash body and a purging liquid solution is pumped through the probe into the chamber. A cleaning liquid solution may also be pumped into the chamber around the probe. Either or both liquids are subsequently vacuumed from the chamber drawing air through an annular gap between the probe and the wash body thereby creating a cleaning air flow between the exterior probe surface and the wash body. The cleaning air flow removes all cleaning liquid solution and/or purging liquid solution as the probe is removed from the wash body.

U. S. Pat. No. 5,813,759 pertains to a vortex mixer which engages and produces a vortex mixing of a liquid within a liquid container by means of centrifugally activated swing-cams. A pair of vertical swing-cams acts to engage the container to the mixer and a horizontal swing-cam provides circular movement to the lower portion of the container. The upper portion of the container is slideably supported so that the container rotates in reaction to the vortex mixing action and thereby produces shear mixing of the liquid.

U.S. Pat. No. 5,776,784 discloses means for separating magnetic particles used in immunoassays from a liquid dispersion disposed in a plurality of reaction vessels, and transporting the reaction vessels in sequence past at least one processing position. A robotics reagent arm and probe dispense reagents into the reaction vessels and a reaction monitoring device is capable of relative movement with respect to the transporting device. Incomplete separation is effected by positioning a magnetic field in contact with the reaction vessel for a first shortened time interval during which the particles partially aggregate and afterwards are removed from the reaction vessel. The magnet is repositioned in contact with the reaction vessel for a third time interval to achieve full separation of particles from the liquid.

U.S. Pat. No. 5,681,695 pertains to a method for increasing specificity in competitive immunoassays by the addition of a reducing agent in the immunoassay. In a one-step assay, the sample, labelled reagent, solid phase and the reducing agent are added simultaneously or in diluents for the sample, labelled reagent or solid phase. In a two-step assay, the sample and solid phase are incubated together-before the addition of the labelled reagent. The reducing agent is preferably added to the sample prior to addition of the solid phase or simultaneously with the sample and solid phase.

U.S. Pat. No. 5,635,364 pertains to a method for verifying that an assay methodology is properly performed, that assay reagents employed possess the necessary potency for accurately performing such assay methodology, and whether or not test samples or assay reagents have been tampered with or are adulterated, is described. The method is performed by employing an assay verification sample, comprising a positive analyte component and the test sample under analysis, wherein the assay verification sample is analyzed employing the same assay reagents and essentially the same assay methodology employed to analyze the test sample.

From this study of the different approaches taken in the prior art, it is apparent that much effort has been given to the challenges encountered with automated processing of complex immunoassays, including the challenges of maintaining high throughput and analytical accuracy. However, what has been overlooked in the prior art is that irregardless of the emphasis placed on the accuracy, precision and throughput of immunoassays, some of the largest potential sources of error concern specimen collection, handling methods and even the way the patient is handled before the specimen is taken.

For example, if a patient's transferrin level is measured before surgery and after surgery, changes in levels can occur simply as a result of postsurgical stress and such changes might lead to erroneous conclusions that would not have been reached if an original sample had been available for retesting. In this instance, transferrin can fall after about 3 hours and ferritin starts to rise shortly afterwards. Thyroid hormone levels are also often repressed after surgery.

The dietary state of an individual may also lead to conclusions that would not have been reached if an original sample had been available for retesting. It is known that lipid levels change after a fatty meal; liver enzymes are affected by alcohol intake; the renin-aldosterone-angiotensin system is strongly affected by posture; and oral contraceptives have a pronounced effect on many binding proteins including those for thyroxine and cortisol.

Errors in interpretation of immunoassay results may also occur if a second patient specimen is not collected correctly. A specimen taken from the side on which a mastectomy has been recently done may not be as equally representative of a patient's health condition because of lymphostasis. In other instances, if a second patient specimen is taken by needle and a primary sample tube used having a rubber stopper made of a plastic such as tris (2 butoxy-ethyl), the stopper itself may cause displacement of some drugs and other analytes from protein binding sites with consequent redistribution between erythrocytes and plasma. Furthermore, the vagaries involved in urine sample collection are well known.

Most systems available today for automated storage and retrieval of patient specimens are based on Total Laboratory Automation (TLA). TLA systems utilize a conveyor system to transport the primary sample tube around the lab from instrument to instrument and then stores the tube in a huge refrigerator for future access. This concept is expensive and requires a significant amount of floor space to achieve.

A Storage Retrieval and Disposal System called SRS, produced by CRS Robotics Corporation, is a large stand-alone, automated system that archives primary sample tubes and retrieves them on request. An operator is required to take the sample to the analytical instrument and schedule the add-on tests.

### SUMMARY OF THE INVENTION

Such failures as these in the prior art to ensure that the same patient specimen is tested a second time following a previous first testing is overcome by using the apparatus and method of this invention. This invention provides a method to automatically reprocess a sample aliquot retained in storage on an automated clinical analyzer for a predetermined period of time in environmentally controlled conditions. Incoming specimens to be tested may be identified by bar coded indicia to determine if a sample aliquot is to be retained, and if so, for what period of time. In addition to a first sample aliquot taken from a patient's specimen to be tested, in accordance with a first embodiment of the present invention, a second sample aliquot is also taken from the same patient's specimen and is retained in a storage compartment within the analyzer. If it becomes desirable to re-test or additionally test a patient's specimen some period of time after tests on the first sample aliquot are completed, reported, and analyzed by a physician, the second sample aliquot may be quickly removed from storage and tested on the analyzer, thereby saving time as well as providing for the exact same patient specimen to be tested. In another embodiment of the present invention, one or more sample aliquots are taken from the a patient's specimen and, after presentation to the analyzer for analysis, the one or more sample aliquots are retained in a storage compartment within the analyzer. If it becomes desirable to later test the patient's specimen, the one or more sample aliquots may be quickly removed from storage and tested on the analyzer. Such novel methods as provided by this invention make it possible to minimize if not totally eliminate the potential sources of error that exist in repeated specimen collection.

### BRIEF DESCRIPTION OF THE DRAWINGS

The invention will be more fully understood from the following detailed description thereof taken in connection with the accompanying drawings which form a part of this application and in which:

FIG. 1 is a schematic plan view of an automated analyzer in which one embodiment of the present invention may be used to advantage;

FIG. 2 is a schematic plan view of an automated analyzer in which another embodiment of the present invention may be used to advantage;

FIG. 3A is a side elevation view of an aliquot storage vessel useful in practicing the embodiment of FIG. 1; and,

FIGS. 3B and 3C are plan views of alternate sample aliquot strips useful in practicing the embodiment of FIG. 2.

### DETAILED DESCRIPTION OF THE INVENTION

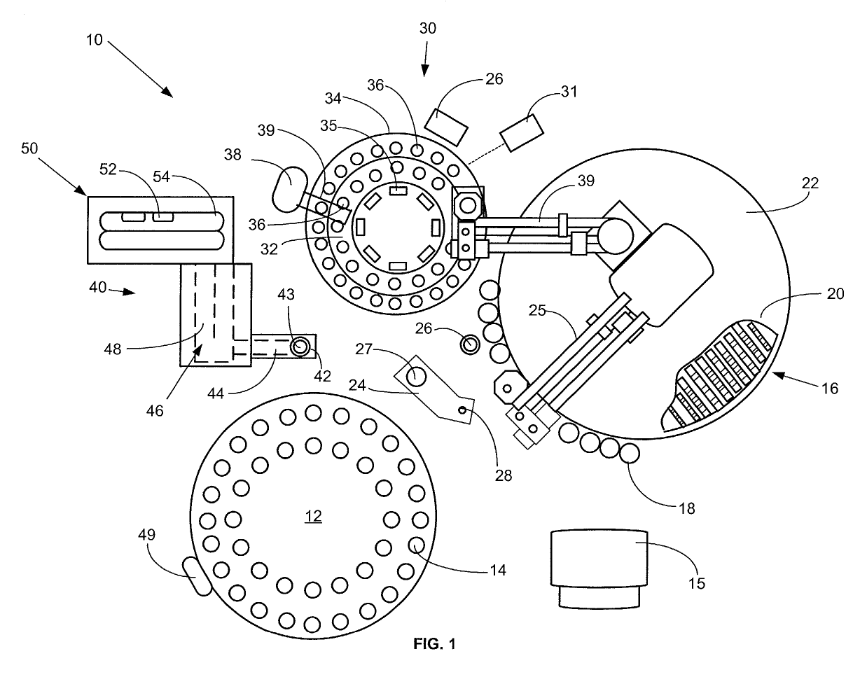

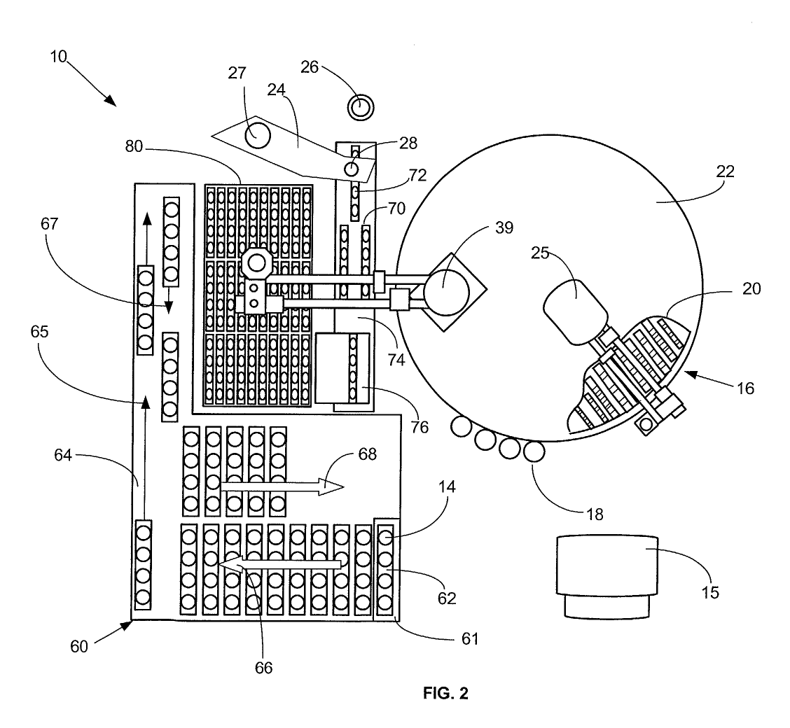

The method and apparatus of this invention will be described initially with particular reference to FIGS. 1 and 2 of the drawings. FIG. 1 shows schematically the elements of a conventional automatic chemical analyzer 10 comprising a sample cup carousel 12 supporting a plurality of open sample tubes 14, a test cuvette carousel 16, adapted to hold a plurality of test cuvettes 18 and to provide plurality of reagent liquid cartridges 20, illustrated as disposed beneath a cut out portion 21 of a lid 22, which covers various thermally controlled compartments. Reagent cartridges 20 may be, for example, a multi-compartment container such as those sold under the tradename FLEX.™. by Dade Behring Inc., Deerfield, Ill. Cuvettes 18 may be formed, as done on the Dimension.RTM. chemical analyzer also sold by Dade Behring Inc., Deerfield, Ill., by pulling two different composition ribbons of clear film from a cuvette film cartridge, not shown, onto the periphery of the cuvette carousel 16. The cuvette carousel 16, preferably in the form of a wheel, has about a hundred separate cavities for holding cuvette 18, the inner wall of each cavity having an opening to allow transmission of light. A small opening remains at the top of each cuvette 18 to allow the addition of reagent liquid and sample liquid. A sample liquid arm 24 and a wash resource 26 used to clean the probe 28 are located proximate the sample cup carousel 12 and cuvette carousel 16. Sample liquid arm 24 supports a conventional sample liquid probe 28 and is mounted to a rotatable shaft 27 so that movement of sample liquid arm 24 describes an arc intersecting the sample cup carousel 12, cuvettes 18, the wash resource 26 as well as an aliquot deposit port 42, described hereinafter. Sample liquid probe 28 is conventionally adapted, for example by cooperation with a peristaltic pump vacuum source, to withdraw from sample tubes 14 all of or aliquot portions of a patient's specimen to be tested by analyzer 10.

A first liquid probe 25 is rotatably mounted above cuvette carousel 16 and is adapted to draw reagent liquid from an appropriate reagent liquid cartridge 20 and deposit each reagent liquid within a predetermined cuvette 18 for processing by the chemical analyzer 10. Probe 25 further comprises an ultrasonic mechanism used for aspirating, dispensing and mixing reagents similar to that used in the Dimension.RTM. chemical analyzer. Since the hydrating, aspirating, dispensing and mixing mechanisms are well known in the art they need not be described further. Photometic analyzing means, not shown, located beneath the cuvette carousel 16 measures light absorbence through the cuvettes 18 at various wavelengths, from which the presence of analyte in the sample liquid may be determined using well-known analytical techniques. Thus far, the chemical analyzer is conventional and may be, for example, the Dimension.RTM. clinical analyzer sold by Dade Behring Inc., Deerfield, Ill., or another similar analyzer commercially available to clinical laboratories.

The Dimension.RTM. clinical analyzer includes a pre-assay sample processing module 30. This facilitates the several additional steps necessary to perform heterogeneous assays without reducing the ability of the chemical analyzer to maintain a high sample throughput. The processing module 30 permits processing either or both of the sample liquid with analyte and/or the reagent liquid, before they are provided to a cuvette 18 for measurement. Sample processing module 30 comprises two pre-assay sample treatment carousels 32 and 34. These are an inner processing carousel 32 and an outer incubation carousel 34, housed in a thermal chamber, (not shown), the two carousels being concentrically mounted with a common axis and preferably lying in a common plane, both preferably being in the form of a circular carousel. Both carousels are independently moveable and have a predetermined number of vessel holding means to support a plurality of individual pre-assay reaction vessels 36.

Drive means 31 are provided for independently rotating incubation carousel 34 and processing carousel 32 about a common axis, the drive means typically comprising gear teeth disposed on each of the carousels 32 and 34 and interlacing with pinion gears mounted on the shaft of a motor (not shown). The drive means may be of conventional design. The transfer station 38 described above is one of the plurality of processing stations.

The incubation carousel 34 contains forty to fifty discrete positions, and is situated to allow reaction vessels 36 to be presented for: 1) reagent addition, 2) sample addition/aspiration, and 3) transfer to/from the cuvette and processing carousels 16, 32, and for load/unload. The carousel may be about 10 inches in diameter. The incubation carousel 34 is also driven via a drive means 31 and uses a single home sensor. Its position can be verified at any time via an encoder attached to the stepper motor. The incubation carousel 34 is slotted to allow vessels to be transferred on/off the carousel horizontally.

When vessels 36 are on the incubation carousel 34, they move inside a thermal incubation trough, which guides the vessels as they travel around the carousel, and keeps them at a steady temperature. The incubation trough is aluminum and heated via a resistive element. A thermistor senses the metal temperature nearest the vessels.

The incubation carousel operation is asynchronous; i.e., it can position any of the vessels to any of three locations as noted above at any time. This provides flexibility in assay formats and complete random access. The processing carousel 32 contains 15 discrete positions, and is situated concentric inside the incubation carousel. The processing carousel allows the vessels to be presented for: 1) magnetic separation, 2) aspirate/wash, 3) re-suspension mixing, and 4) transfer on/off the processing carousel to the incubation carousel.

The processing carousel 32 is driven by the drive means 31, by the same as the incubation carousel. Similar to the incubation carousel, the processing carousel is slotted to allow vessels to be transferred on/off. The vessels are held in place on the carousel with spring clips. Unlike the incubation carousel, the sequencing of the processing carousel is synchronous or repetitive. Whenever reaction vessels 36 are present on the carousel, the carousel will index in a rote manner, advancing each vessel through a series of separate-wash-mix steps.

A common transfer station 38, which accesses both carousels 32 and 34, is provided for transferring reaction vessels 36 between the two carousels 32 and 34 and for removing reaction vessels 36 from the sample processing module 30 and passing them into a suitable waste disposal, not shown. The transfer station 38, which may be of conventional design, is used to transfer reaction vessels 36 to/from the processing carousel and to load/unload vessels from the incubation carousel 34. New vessels are routed to the vessel transfer station 38 via a feedtrack 44. Used vessels are routed to the waste container via a chute attached to the underside of the transfer station, beneath a hole in the exit track (not shown).

A second liquid probe 39 is rotatably mounted above cuvette carousel 16 and is adapted to draw reagent liquid from an appropriate reagent liquid cartridge 20 and deposit such reagent liquid in a predetermined reaction vessel 36 in the incubation carousel 34. Sample liquid probe 28 is also adapted (1) to draw sample liquid from a reaction vessel 36 after the sample liquid has undergone the scheduled pre-assay operations and (2) to deposit sample liquid within a predetermined cuvette 18 for further processing and measurement.

Sample processing devices, or stations 35, are positioned at selected circumferential locations about the processing carousel 32 such that they can access reaction vessels 36. It will be recalled the processing carousel 32 is concentrically mounted with the incubating carousel 34, radially outside of the processing carousel 32 (depicted in FIG. 1 as inside for the sake of clarity). These stations are adapted to provide for mixing together of the sample liquid and the reagent liquid contained in a reaction vessel 36, for washing the sample liquid and the reagent liquid contained in a reaction vessel 36, and for magnetic separation of tagged magnetic particles from free tags or reagent liquid contained in a pre-assay reaction vessel 36.

The present invention adds to analyzer 10 or similar analyzers available to clinical laboratories a method to automatically and quickly test a second sample aliquot retained in storage for a predetermined period of time in environmentally controlled conditions on analyzer 10. Incoming specimens to be tested are identified by reading with a conventional bar code reader 49 bar coded indicia on sample tubes 14 to determine, among other items, a patient's identity, the tests to be performed, if a sample aliquot is desired to be retained and if so, for what period of time. In addition to a first sample aliquot taken by sample liquid probe 28 from sample tubes 14 containing the specimen to be tested, a second sample aliquot may also taken by sample liquid probe 28 from the specimen and this second sample aliquot is retained by analyzer 10 within an environmentally controlled storage compartment 50.

This present invention thus provides sample retention and transfer means 40 comprising an aliquot deposit port 42 to receive a second sample aliquot from sample liquid probe 28, an open aliquot storage vessel 43 to retain said second sample aliquot, vessel transfer means 46 to move storage vessels as directed between aliquot deposit port 42 and environmentally controlled storage compartment 50. Vessel transfer means 46 may take on any number of features, for example moving belts 44 and/or robotic devices 48, adapted to move a storage vessel 43 between aliquot deposit port 42 and storage compartment 50. Storage compartment 50 comprises a closed region having an interior portion and well known humidity and temperature control devices (not shown) to maintain the interior portion of the storage compartment 50 at temperatures between minus 4 degrees Centigrade and plus 20 degrees Centigrade and relative humidity between about 5% and 75%.

Storage compartment 50 further comprises inventory storage means 52, for example shelves, clips, or other similar storage vessel receptacles 52 adapted to securely support a storage vessel 43. Storage compartment 50 also comprises inventory warehousing means 54, for example serpentine belts or tracks or revolving racks 54 adapted to support vessel receptacles 52. A key feature of inventory storage means 52 and inventory warehousing means 54 is the ability to re-present any vessel receptacle 52 maintained within storage compartment 50 to Vessel transfer means 46 so that Vessel transfer means 46 may represent a storage vessel 43 to sample liquid probe 28.

This present invention also provides for various methods to determine the period of time a second sample aliquot is retained in an aliquot storage vessel 43 within environmentally controlled storage compartment 50.

This present invention also provides a method to aliquot and store QC products and Calibrators in order to perform automated QC and Calibration. In carrying out immunoassay procedures for determining concentrations of analytes, a common practice is to use a family of controlled formulation solutions, hereinafter called QC products Calibrators, each of which contains accurately predetermined quantities or concentrations of various analytes. Concentrations that are substantially lower and higher than normal are generally employed. Since the immunoassay procedures are normally designed to analyze serum samples, it is preferred that the calibration solutions be formulated using a liquid matrix that is identical to or equivalent to serum, thereby also avoiding the possibility of inaccurate rehydration of lyophilized calibration materials. Bottles of QC products and Calibrator could be presented to sample liquid probe 28 and individual aliquots removed until the supply was empty. The original QC products and Calibrators could be stored onboard analyzer 10 in storage compartment 50 until their expiration date is reached.

In a first embodiment, a second sample aliquot is taken by sample liquid probe 28 from every patient specimen placed in a sample cup 14 and is retained in an aliquot storage vessel 43 within environmentally controlled storage compartment 50 for a first predetermined period of time, for example two weeks, after tests on the corresponding first sample aliquot is taken by sample liquid probe 28 are completed. In this instance, at any time during said first predetermined period of time that a request was made to repeat a test or to perform additional tests on the previously tested patient specimen, this request is presented to analyzer 10 either by an operator or automatically by a Laboratory Information System electronically connected to analyzer 10. The operating computer CPU 15 of analyzer 10 subsequently provides appropriate commands to inventory storage means 52 and inventory warehousing means 54 to remove the vessel receptacle 52 containing the second aliquot of the previously tested patient specimen and to present said second aliquot of the previously tested patient specimen to vessel transfer means 46 and similarly commands transfer means 46 to present storage vessel 43 to aliquot deposit port 42. At this stage, as previously described, sample liquid arm 24 supporting sample liquid probe 28 is moveable by rotatable shaft 27 to bring sample liquid probe 28 to aliquot deposit port 42 where a portion or all of second sample aliquot is aspirated by sample liquid probe 28. Next, sample liquid arm 24 is moveable by rotatable shaft 27 to bring sample liquid probe 28 to one or more cuvettes 18 as required to complete the requested tests on the previously tested patient specimen and to deposit a portion or all of second sample aliquot into said cuvettes 18. Operation of analyzer 10 then proceeds as described above to complete the tests requested on the second sample aliquot of the previously tested patient specimen, without requiring that a second patient specimen be obtained.

In a second embodiment, a second sample aliquot is taken by sample liquid probe 28 only from those sample tubes 14 that have bar code indicia containing instructions to retain such a second sample aliquot on-board analyzer 10 within an environmentally controlled storage compartment 50. In this second embodiment, the bar code indicia may also containing instructions that establish the particular period of time that the second sample aliquot is retained in an aliquot storage vessel 43 within environmentally controlled storage compartment 50 after tests on the corresponding first sample aliquot are completed. Obviously, in this second embodiment, the bar code indicia may simply instruct that the second sample aliquot be retained within environmentally controlled storage compartment 50 for some standard period of time, for example, two weeks, as was done in the previously described first embodiment. Similarly to the first embodiment, at any time during the period of time that the second sample aliquot is retained within storage compartment 50, a request to repeat a test or to perform additional tests on the previously tested patient specimen, is automatically performed by analyzer 10, without requiring that a second patient specimen be obtained.

In a third embodiment, a second sample aliquot is taken by sample liquid probe 28 only from those sample tubes 14 that have bar code indicia containing instructions for analyzer 10 to perform certain analytical tests or groups of tests and the bar code indicia do not contain instructions either to store or redarding a specific time to store such a second sample aliquot on-board analyzer 10. In this third embodiment, computer CPU 115 contains a look-up-table in memory that automatically establishes from the analytical tests or groups of tests requested the particular period of time that the second sample aliquot is retained in an aliquot storage vessel 43 within environmentally controlled storage compartment 50 after tests on the corresponding first sample aliquot are completed. For example, if a Standard Metabolic Panel (CHEM 8) including Na, K, Cl, CO2, GLUC, BUN, CREA, and CA is to be performed, the second sample aliquot may be automatically retained in an aliquot storage vessel 43 for a two week period of time.

In another instance, if the original patient specimen is to be tested for indications of abnormal levels of drugs of abuse or prostrate specific antigen, tests that may be done as part of a routine employment examination or to diagnose a highly specific disease, the period of time that the second sample aliquot is retained in storage compartment 50 may be as short as one or two days, since no additional or repeated testing is expected. In contradistinction, if the original patient specimen is to be tested for indications of abnormal PSA levels, a test that is not done as part of a routine examination, the period of time that the second sample aliquot is retained in storage compartment 50 may be as long as one or two weeks, since additional or repeated testing may be expected as part of a full diagnosis. Similarly to the above first and second embodiments, at any time during the period of time that the second sample aliquot is retained within storage compartment 50, a request to repeat a test or to perform additional tests on the previously tested patient specimen, is automatically performed by analyzer 10, without requiring that a second patient specimen be obtained.

In a fourth embodiment, a second sample aliquot is taken by sample liquid probe 28 from every patient specimen placed in a sample cup 14 and is retained in an aliquot storage vessel 43 within environmentally controlled storage compartment 50 for a relatively short period of time, for example two days, after tests on the corresponding first sample aliquot is taken by sample liquid probe 28 are completed. In this embodiment, the purpose of storing a second sample aliquot from every patient specimen is to allow for unusually large variances from normally expected tests results that might occur, for instance, as a result of unrecognized operator error or reagent or analyzer failure.

In all of these embodiments, when the particular period of time has elapsed that the second sample aliquot is retained within storage compartment 50 after tests on the corresponding first sample aliquot are completed, then operating computer CPU 15 of analyzer 10 subsequently provides appropriate commands to inventory storage means 52 and inventory warehousing means 54 to remove the vessel receptacle 52 containing the second aliquot of the previously tested patient specimen and to dispose of said second aliquot into a trash dump (not shown) provided as part of storage compartment 50.

Another key feature of inventory storage means 52 and inventory warehousing means 54 is the ability to store storage vessels 43 at locations that facilitate rapid retrieval of a given storage vessel 43 from storage compartment 50. As previously described, the period of time that sample aliquots are is retained within storage compartment 50 may be different for different patient samples. Periods of time, for example like from 2 days to 2 weeks, may be dictated by either analyzer 10 or by specific instructions that accompany the incoming patient sample. Thus, storage compartment 50 may advantageously be arranged so that patient samples having shorter periods of time to be retained within storage compartment 50 may be more quickly accessed by vessel transfer means 46. Such an arrangement may be enabled by storing all storage vessels 43 having shorter periods of storage time at locations most proximate within storage compartment 50 to vessel transfer means 46. Alternately, storage compartment 50 may advantageously be arranged so that patient samples having the same future date to be automatically removed from within storage compartment 50 and disposed into a trash receptacle may be stored at one contiguous location within storage compartment 50 to expedite such a trashing operation.

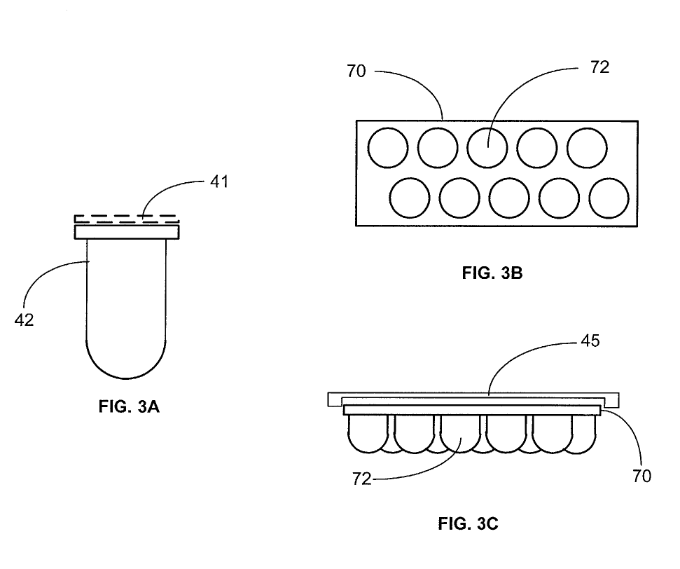

The aliquot storage vessel 43 containing the second aliquot of the previously tested patient specimen can take any of several relatively equivalent variations as long as an opening is available to deposit liquid sample into and extract liquid sample from aliquot storage vessel 43 using sample liquid probe 28 or its equivalent. An exemplary aliquot storage vessel 43 is illustrated in FIG. 3A. It should be understood that the aliquot well opening(s) of aliquot storage vessel 43 or of a sample aliquot strip 70, described later, may optionally be covered with a layer 41 of protective film (shown in dashed lines in FIG. 3A) that does not hinder subsequent probe puncture after liquid probe 28 has deposited the second aliquot of the previously tested patient specimen therein. The layer 41 of protective film may alternately comprise a thin layer of a heat sealed plastic or foil or a thin layer of a plastic or foil having adhesive on one surface or a lid 45 of some kind (see FIG. 3C) that can be applied and removed or easily pierced.

FIG. 2 illustrates another embodiment of the present invention in which patient sample tubes 14 held in a plurality of sample tube racks 62 may be moved, for example in the directions indicated by arrows 65 and 67, by a sample tube rack transport system 60 over a base portion 64 of analyzer 10 from a tube rack loading zone 61 to sample liquid probe 28. When a sample tube 14 is proximate sample liquid probe 28, as described previously sample liquid probe 28 is adapted to aspirate sample liquid from sample tubes 14, in this embodiment, a relatively large volume of sample liquid, in the range of about 100 μL to 500 μL is removed from sample tubes 14. Subsequent to aspiration of sample liquid, sample liquid probe 28 may be rotated by shaft 27 to a location above a sample aliquot strip 70 having a number of open aliquot wells 72 therein. In this embodiment, sample liquid probe 28 is controllable by CPU 15 to deposit a like quantity of sample liquid into each of the open aliquot wells 72. Analyzer 10 includes a second liquid probe 39 adapted to remove whatever portion of sample liquid is required to perform the assays prescribed for the corresponding particular patient specimen and to present said sample liquid to the various processing stations for analysis as described above. A sample aliquot strip transport system 74 is adapted to move aliquot strips 70 from proximate sample liquid probe 28 to an aliquot strip pre-storage system 76 having means therein to seal the openings of aliquot wells 72, to apply identifying indicia to aliquot strips 70 and to transfer aliquot strips 70 into an environmentally controlled storage compartment 80. Sample aliquot strips 70 may have, for example, three separate open aliquot wells 72 therein.

In a manner similar to described previously relative to storage compartment 50, storage compartment 80 comprises aliquot strip storage means 82, for example shelves, clips, or other similar storage receptacles 82 adapted to securely support an aliquot strip 70. Storage compartment 80 also comprises inventory warehousing means 84, for example serpentine belts or tracks or revolving racks 84 adapted to support aliquot strips 70. A key feature of inventory storage means 82 and inventory warehousing means 82 is the ability to re-present any aliquot strip 70 maintained within storage compartment 80 to aliquot strip transport system 74 so that aliquot strip transport system 74 may re-present an aliquot strip 70 to second liquid probe 39 rotatably mounted above cuvette carousel 16 and adapted to draw reagent liquid from an appropriate aliquot strips 70 and deposit such reagent sample in a predetermined reaction vessel 18 in the incubation carousel 34.

The aliquot strip 70 containing multiple aliquots of patient specimen can take any of several relatively equivalent variations as long as at least one open well is available to deposit liquid sample into and extract liquid sample from using sample liquid probe 28 or its equivalent. Two exemplary but alternate aliquot strips 70 are illustrated in FIGS. 3B and 3C.

It is to be understood that the embodiments of the invention disclosed herein are illustrative of the principles of the invention and that other modifications may be employed which are still within the scope of the invention. For instance, aliquot strip 70 having a number of open aliquot wells therein may be used in the first embodiment of the present invention (FIG. 1), and similarly, the aliquot storage vessel 43 may be used in the second embodiment of the present invention (FIG. 2), both variations still achieving a primary object of the present invention of providing a method to additionally test a patient's specimen some period of time after tests on an aliquot portion taken from the patient's specimen are completed by retaining said aliquot of the patient's specimen within a clinical analyzer for a period of time. Accordingly, the present invention is not limited to those embodiments precisely shown and described in the specification as available on the Dimension.RTM. chemical analyzer but only by the following claims.

What is claimed is:

1. A method to additionally test a patient's specimen using an analyzer some period of time after tests on an aliquot portion taken from the patient's specimen are completed by retaining said aliquot of the patient's specimen within said analyzer for a period of time.

2. A method to additionally test a patient's specimen using an analyzer some period of time after tests on a first aliquot portion taken from the patient's specimen are completed by retaining a second aliquot portion taken from the patient's specimen within said analyzer for a period of time.

3. The method of claim 1 wherein the patient's specimen is retained within a storage compartment.

4. The method of claim 2 wherein the patient's specimen is retained within a storage compartment.

5. The method of claim 3 wherein the storage compartment comprises environmentally controlled conditions.

6. The method of claim 4 wherein the storage compartment comprises environmentally controlled conditions.

7. The method of claim 1 wherein the patient's specimen is marked to determine if an aliquot of a patient's specimen is to be retained in storage.

8. The method of claim 2 wherein the patient's specimen is marked to determine if an aliquot of a patient's specimen is to be retained in storage.

9. The method of claim 1 wherein the patient's specimen is marked to determine the period of time for patient's specimen to be retained in storage.

10. The method of claim 2 wherein the patient's specimen is marked to determine the period of time for patient's specimen to be retained in storage.

11. The method of claim 1 wherein tests to be performed upon a patient's specimen are examined to ascertain the period of time a patient's specimen is to be retained in storage.

12. The method of claim 2 wherein tests to be performed upon a patient's specimen are examined to ascertain the period of time a patient's specimen is to be retained in storage.

13. The method of claim 11 wherein the patient's specimen is examined by the analyzer.

14. The method of claim 12 wherein the patient's specimen is examined by the analyzer.

15. The method of claim 1 wherein the patient's specimen is positioned in storage in accord with the expiration of the period of time.

16. The method of claim 2 wherein the patient's specimen is positioned in storage in accord with the expiration of the period of time.

17. The method of claim 1 wherein the patient's specimen is positioned in storage in accord with the length of the period of time.

18. The method of claim 2 wherein the patient's specimen is positioned in storage in accord with the length of the period of time.

19. The method of claim 1 where the aliquots of the patient's specimen are retaining in a aliquot strip having a number of open aliquot wells therein.

20. The method of claim 2 where the aliquots of the patient's specimen are retaining in a aliquot strip having a number of open aliquot wells therein.

21. The method of claim 2 where the aliquots of the patient's specimen are retaining in a open aliquot storage vessel.

22. The method of claim 1 wherein the patient's specimen to be stored is covered with layer of protective film.

23. The method of claim 2 wherein the patient's specimen to be stored is covered with layer of protective film.

24. The method of claim 22 wherein the layer of protective film is a thin layer of a heat sealed plastic or foil.

25. The method of claim 23 wherein the layer of protective film is a thin layer of a heat sealed plastic or foil.

26. The method of claim 22 wherein the layer of protective film is a thin layer of a plastic or foil having adhesive on one surface.

27. The method of claim 23 wherein the layer of protective film is a thin layer of a plastic or foil having adhesive on one surface.

28. The method of claim 22 wherein the layer of protective film is a lid that can be applied and removed or easily pierced.

29. The method of claim 23 wherein the layer of protective film is a lid that can be applied and removed or easily pierced.

30. A method to automatically extract an initial portion of a formulation solution having an expiration date onboard an analyzer and to retain said formulation solution in storage onboard said analyzer until the expiration date of the formulation solution is reached.

31. The method of claim 30 further comprising removing said formulation solution from storage and extracting a subsequent portion of said formulation solution before the expiration date of the formulation solution is reached.

32. The method of claim 30 wherein the formulation solution is stored in an environmentally controlled compartment.
